(19) United States
(12) Patent Application Publication
(10) Pub. No.: US 2010/0029944 A1
(43) Pub. Date: Feb. 4, 2010

(54) Process for the Synthesis of Solifenacin

(75) Inventors: Jordi Puig, Girona (ES); Laura Sanchez, Girona (ES); Ester Masllorens, Girona (ES); Ignasi Auger, Girona (ES); Jordi Bosch, Girona (ES)

Correspondence Address: COHEN, PONTANI, LIEBERMAN & PAVANE LLP, 551 FIFTH AVENUE, SUITE 1210, NEW YORK, NY, 10176, US

(73) Assignee: Corporacion Medichem, S.L., Sant Joan Despi, Barcelona (ES)

(21) Appl. No.: 12/515,689

(22) PCT Filed: Nov. 20, 2007
(86) PCT No.: PCT/IB2007/003569

§ 371 Date: Aug. 4, 2009

Related U.S. Application Data
(60) Provisional application No. 60/860,547, filed on Nov. 22, 2006.
(60) Provisional application No. 60/903,927, filed on Feb. 28, 2007.

(51) Int. Cl. C07D 453/02; C07D 401/12

(52) U.S. Cl. 546/137

(57) ABSTRACT

This invention provides improved methods for making solifenacin and pharmaceutically acceptable salts thereof. The instant methods are unexpectedly advantageous in their simplicity and efficiency.

This application claims priority of U.S. Provisional Application Nos. 60/860,547, filed Nov. 22, 2006, and 60/903,927, filed Feb. 28, 2007, the contents of which are incorporated herein by reference in their entireties.

Throughout this application, various publications are cited. The disclosure of these publications is hereby incorporated by reference into this application to describe more fully the state of the art to which this invention pertains.

### BACKGROUND OF THE INVENTION

Solifenacin succinate is a commercially marketed pharmaceutically active substance indicated for the treatment of overactive bladder with symptoms of urinary incontinence, urgency and high urinary frequency. Solifenacin succinate is the international common denomination for butanedioic acid compounded with (1S)-(3R)-1-azabicyclo[2.2.2]oct-3-yl-3,4-dihydro-1-phenyl-2(1H)-isoquinolinecarboxylate (1:1), having an empirical formula of C₂₃H₂₆N₂O₂.C₄H₆O₄ and the structure represented in formula I given below.

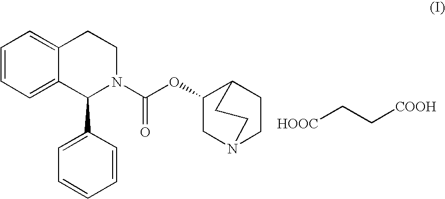

Solifenacin and its pharmaceutically acceptable salts are reported in U.S. Pat. No. 6,017,927 (the '927 patent).

The following Scheme 1 shows the synthetic routes disclosed in the '927 patent for the preparation of (1RS, 3′RS)-solifenacin and (1S,3′RS)-solifenacin:

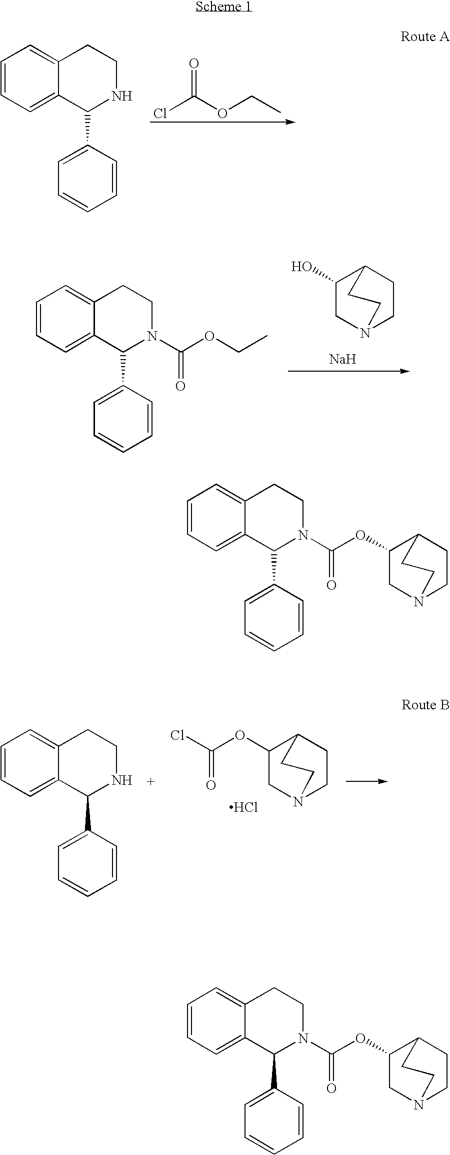

The following Scheme 2 shows the synthetic route disclosed in WO2005075474 for the preparation of solifenacin and solifenacin succinate:

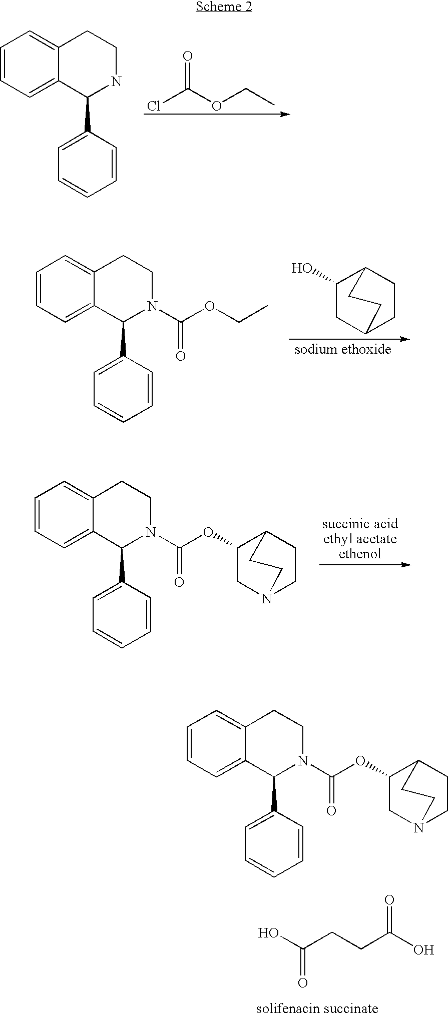

Patent application WO2005/105795A1 discloses, among other things, an improved process for preparing solifenacin, which is represented in Scheme 3 below, wherein Lv can be 1H-imidazole-1-yl or chloride, using sodium hydride as a base and a mixture of toluene and dimethylformamide or toluene alone as an organic solvent.

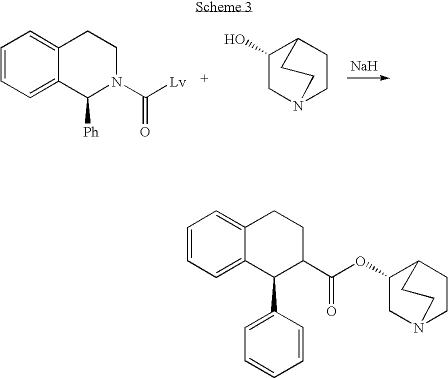

However, this process presents some drawbacks. First, toluene and DMF are listed as Class 2 solvents by the ICH (International Convention on Harmonisation, a tri-regional organization that represents the drug regulatory authorities of the European Union, Japan and the United States), which means that they are associated with significant toxicity. Accordingly, they are listed as solvents to be limited in order to protect patients from potential adverse effects. Further, in order to meet with the limits of residual solvents of the ICH for toxic solvents, the solifenacin obtained by this process shall not exceed a concentration limit of 890 ppm and 880 ppm for toluene and DMF, respectively.

Second, the work-up process is laborious and makes use of a large number of liquid-liquid extraction processes, which may decrease the efficiency of the process.

Processes described in the prior art for the preparation of solifenacin and solifenacin succinate are not very efficient or suitable for industrial scale-up because they include a laborious work-up with operations such as distillations, chromatographic purifications or large number of liquid-liquid extraction processes. Further, most of these processes use reaction solvents that are associated with significant toxicity. So there is a need for an improved and simplified process for the preparation of solifenacin and/or one of its salts.

Further, the preparation of solifenacin succinate has been explicitly described in patent application WO2005/075474A1. Examples 1, 2 and 3 of WO2005/075474A1 describe the preparation of solifenacin succinate by reacting solifenacin and succinic acid in ethanol and ethyl acetate as solvents. However, the use of ethanol in this preparation presents an important drawback, i.e. ethanol may undergo esterification reaction in the presence of succinic acid, which hence may decrease the efficiency of the process. Furthermore, W02005/075474A1 does not describe certain key factors for efficiently preparing solifenacin succinate, such as the time required for dissolving the reaction mixture as well as the time required for the solifenacin succinate salt to precipitate efficiently. In this regard, succinic acid is poorly soluble in the majority of organic solvents, and therefore its solution requires amounts of polar organic solvents (e.g. ethanol) which consequently make the precipitation of the final solifenacin succinate salt troublesome. Accordingly, the preparation of solifenacin succinate generally becomes an arduous task which makes use of extensive preparation time and usually affords the desired product inefficiently.

Example 1A of Patent application WO2005/105795A1 also discloses a preparation of solifenacin succinate from a mixture of solifenacin, ethanol, ethyl acetate and succinic acid. However, the preparation of solifenacin succinate by that method requires a total time of 7 hours. Namely, the reaction mixture must be heated at 50° C. for 2 hours and then cooled to 0° C. requiring 5 hours. In this regard, long-time reactions may represent an important drawback for industrial implementation, especially in terms of reactor occupation time.

In view of the foregoing there is also a need for a shorter, more efficient and simplified processes for the preparation of solifenacin succinate, which are suitable for industrial implementation.

### SUMMARY OF THE INVENTION

The present invention provides an improved synthetic strategy for the preparation of solifenacin and pharmaceutically acceptable salts thereof in a more efficient and simplified way.

Accordingly, a first aspect of the present invention relates to a process for obtaining solifenacin, or a pharmaceutically acceptable acid addition salt, which comprises:

a) reacting a compound of formula II

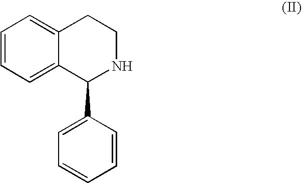

with a compound of formula III

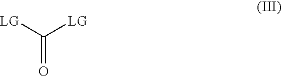

wherein LG represents 1H-imidazole-1-yl, 4-methyl-[1,2,4]oxadiazolidine-3,5-dione-2-yl, or 1H-1,2,4-triazol-1-yl or CCl₃ to obtain the compound of formula IV

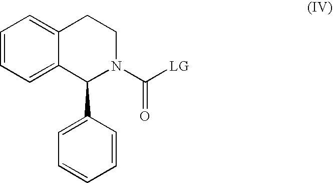

wherein LG represents 1H-imidazol-1-yl, 4-methyl-[1,2,4]oxadiazolidine-3,5-dione-2-yl, 1H-1,2,4-triazol-1-yl or CCl₃ and

b) reacting the compound IV obtained in step (a) with a compound of formula V that is activated by a base to form an alkoxide

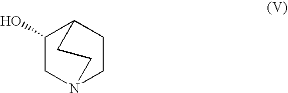

in the presence of a Lewis acid, to give solifenacin (Ia)

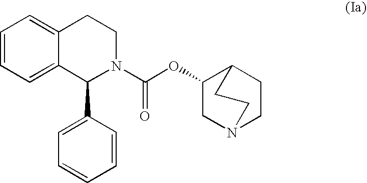

which could then optionally be converted to one of its pharmaceutically acceptable acid addition salts. The preferred Lewis acid is aluminium trichloride. Other Lewis acids include titanium-based catalysts such as titanium isopropoxide. The preferred base is sodium hydride or sodium tert-amyloxide.

In a second aspect, the invention provides a process for converting solifenacin to its succinate salt comprising adding a solution of solifenacin base in ethyl acetate over a solution of succinic acid in acetone.

In a third aspect, the invention provides crude solifenacin with less than 30% of (S)-1-phenyl-1,2,3,4-tetrahydroisoquinoline.

In a fourth aspect, the invention provides crude solifenacin obtained without isolating the compound of formula IV (wherein LG represents 1H-imidazole-1-yl, 4-methyl-[1,2,4]oxadiazolidine-3,5-dione-2-yl, or 1H-1,2,4-triazol-1-yl or CCl₃).

### DETAILED DESCRIPTION OF THE INVENTION

The present invention provides an improved process for efficiently preparing solifenacin and/or one of its pharmaceutically acceptable salts.

Using the process according to the present invention, solifenacin is obtained in a simplified way, using milder reaction conditions and without the need for laborious operations such as chromatographic purifications or solvent distillations. So the process according to the present invention is very suitable for industrial scale-up. The process for preparing solifenacin succinate salt according to this invention overcomes the drawbacks of the prior art by, inter alia, (i) using a ketone solvent to effectively dissolve succinic acid (which solvent does not undergo unwanted esterification reactions in the presence of succinic acid), and (ii) allowing a rapid (about 2 hours) and efficient precipitation of solifenacin succinate by partially distilling off the solvents of the mixture before the cooling step.

Syntheses of ureas, carbamates and thiocarbamates can be performed by transferring an electrophilic carbamoylating reagent to the corresponding nucleophilic moiety. Solifenacin, as an organic carbamate, can be prepared by reacting a nucleophilic alcohol with the appropriate electrophilic reagent.

Surprisingly, the presence of a Lewis acid such as aluminium trichloride in the reaction medium favours the desired reaction pathway that leads to solifenacin instead of the undesired reaction pathway that leads back to the formation of compound II.

The first preferred embodiment of the present invention is a process for obtaining solifenacin which comprises:

a) reacting a compound of formula II

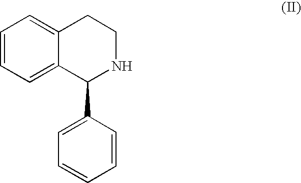

with a compound of formula III

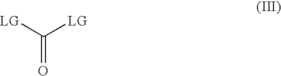

wherein LG represents 1H-imidazol-1-yl, 4-methyl-[1,2,4]oxadiazolidine-3,5-dione-2-yl, 1H-1,2,4-triazol-1-yl or CCl₃, to obtain a compound of formula IV

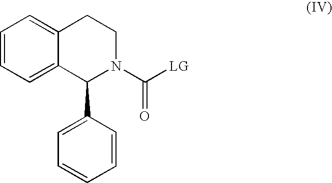

wherein LG represents 1H-imidazole-1-yl, 4-methyl-[1,2,4]oxadiazolidine-3,5-dione-2-yl, 1H-1,2,4-triazol-1-yl or CCl₃ and

b) reacting the compound obtained in step (a) with a compound of formula V that is activated by a base to form an alkoxide

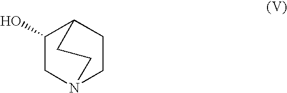

in the presence of aluminum trichloride or a titanium-based catalyst as the Lewis acid, to give solifenacin (Ia)

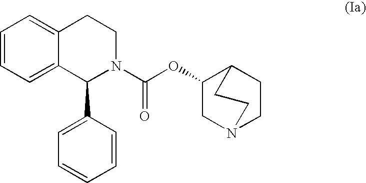

which could then optionally be converted to one of its pharmaceutically acceptable salts.

The second preferred embodiment of this invention is the use of titanium isopropoxide as the Lewis acid.

The third preferred embodiment of the present invention is the use of N,N′-carbonyldiimidazole as a compound of formula III.

The fourth preferred embodiment of the present invention is the use of Bis-[1H-1,2,4-triazol-1yl]-methanone as a compound of formula III.

The fifth preferred embodiment of the present invention relates to the use of 4-methyl-2-[(4-methyl-3,5-dioxo-1,2,4-oxadiazolidin-2-yl)carbonyl]-1,2,4-oxadiazolidine-3,5-dione as a compound of formula III.

The sixth preferred embodiment of the present invention is the use of bis(trichloromethyl)carbonate (triphosgene) as a compound of formula III.

The seventh preferred embodiment of the present invention is a process for obtaining crude solifenacin with less than 30% of (S)-1-phenyl-1,2,3,4-tetrahydroisoquinoline, preferably with less than 20%, less than 10%, less than 5%, less than 2%.

Reaction (a) is conveniently carried out in the presence of an inert organic solvent or a mixture of such solvents. Preferably the solvent is an ether, an aromatic hydrocarbon, an aliphatic hydrocarbon or a chlorinated hydrocarbon. Preferably the chosen solvent is tetrahydrofuran, 2-methyltetrahydrofuran, toluene, xylene, hexane, heptane, cyclohexane, chloroform, dichloromethane, 1,2-dichloroethane, or mixtures thereof. More preferably, the solvent is tetrahydrofuran. The temperature preferably is from about 5° C. to about 40° C. More preferably, the reaction is performed at room temperature.

Reaction (b) is conveniently carried out in the presence of an inert organic solvent from the list above, or a mixture of such solvents. The temperature preferably is from about 0° C. to about the temperature at which the solvent refluxes.

Preferably 1 to 2 equivalents of compound V, and more preferably 1 equivalent, are used to perform reaction (b).

Compounds employed as raw materials or as intermediates to produce solifenacin, can optionally be employed in their free base, salt and/or solvate forms where appropriate.

### EXAMPLES

Reference to HPLC purity is defined by the methods described below:

### HPLC Method for the Assessment of Chemical Purity

The chromatographic separation was carried out in a Phenomenex Luna C18, 5 μm, 4.6 mm×150 mm column.

The mobile phase A was a mixture of 998 ml of 0.010 M ammonium bicarbonate buffer and 2 ml of triethylamine. The pH of the mixture was adjusted to 7.5 with formic acid. Buffer solution was prepared from 0.79 g of NH₄HCO₃ dissolved in 1000 ml of water. The mobile phase was mixed and filtered through a 0.22 μm nylon membrane under vacuum.

The mobile phase B was acetonitrile.

The chromatograph was programmed as follows: Initial 0-2 min. 75% mobile phase A, 2-5 min. linear gradient to 60% mobile phase A, 5-40 min. isocratic 60% mobile phase A, 40-45 min. linear gradient to 75% mobile phase A and 45-50 min. equilibration with 75% mobile phase A.

The chromatograph was equipped with a 220 nm detector and the flow rate was 1.0 ml per minute at 20-25° C. Test samples (20 μl) were prepared by dissolving 20 mg of sample in a mixture of 5 ml of mobile phase A and 5 ml of mobile phase B.

HPLC Method for the Assessment of Optical Purity of compound of Formula II

The chromatographic separation was carried out in a Daicel CHIRALCEL OD-H, 5 μm, 4.6×250 mm column; at 40° C.

The mobile phase was prepared by mixing 500 ml of n-Hexane, 8 ml of Isopropanol and 1 ml of Diethylamine.

The chromatograph was equipped with a 230 nm detector and the flow rate was 1.0 ml/min. Test samples (10 μl) were prepared by dissolving 200 mg of product in 10 ml of diluent. The diluent was prepared by mixing 50 ml of n-Hexane, 50 ml of Isopropanol and 0.2 ml of Diethylamine.

### Example 1

### Preparation of (1S)-(3R)-1-azabicyclo[2.2.2]oct-3-yl-3,4-dihydro-1-phenyl-2(1H)-isoquinolinecarboxylate succinate (solifenacin succinate)

To a cooled solution of N,N′-carbonyldiimidazole (23.1 g, 142.5 mmol) in THF (156 mL), 25.0 g of (1S)-1-phenyl-1,2,3,4-tetrahydroisoquinoline (119.4 mmol) were added and the reaction mixture was stirred at room temperature for 2 h. This solution was finally diluted with 156 ml of THF.

To a mixture of (3R)-3-quinuclidinol (16.8 g, 132.1 mmol), sodium tert-amyloxide (14.6 g, 132.6 mmol) and aluminium chloride (1.1 g, 8.2 mmol), the previously prepared solution was added. The reaction mixture was stirred at reflux for 7 hours and then 150 mL of water were added to distil all the organic solvent. The residue was basified to pH>10 with an aqueous solution of NaOH 50% and stirred for 10-15 minutes. The resulting aqueous phase was extracted with EtOAc (2×130 mL) and the joined organic phases were washed with brine (2×100 mL).

Separately, 14.16 g of succinic acid (120.0 mmol) and 290 mL of acetone were combined in a suitable reactor and heated to reflux until complete dissolution. The previous ethyl acetate solution of solifenacin base was then poured drop-wise to the refluxing succinic acid solution. The mixture was maintained at 55-60° C. for approximately 10-15 minutes with continuous stirring. The reactor was then cooled to room temperature and maintained at 20-25° C. for approximately 1 hour and then was cooled to 0-5° C. for 2 h.

Thereafter, the suspension was filtered, and the collected wet solid was dried under vacuum at 40° C. until constant weight to yield 40.67 g (84.6 mmol, 70.8%) of solifenacin succinate. Analysis by HPLC (area percentage): 98.09% of solifenacin, Titration: 105.89%.

A 23.5 g fraction of the solid obtained was dissolved in 150 mL of water, basified until pH>10 with K₂CO₃ and extracted with EtOAc (2×50 mL). The joined organic phases were poured drop-wise to a refluxing solution containing 5.14 g (43.6 mmol) of succinic acid and 106 ml of acetone. The mixture was maintained at 55-60° C. for approximately 10-15 minutes with continuous stirring. The reactor was then cooled to room temperature and maintained at 20-25° C. for approximately 1 hour and then cooled to 0-5° C. for 2 h. The suspension was filtered, and the collected wet solid was dried under vacuum at 40° C. until constant weight to yield 20.13 g (41.9 mmol, 93.8%) of solifenacin succinate. Analysis by HPLC (area percentage): 99.9% of solifenacin, Titration: 99.72%.

### Example 2

### Preparation of (1S)-(3R)-1-azabicyclo[2.2.2]oct-3-yl-3,4-dihydro-1-phenyl-2(1H)-isoquinolinecarboxylate succinate (solifenacin succinate)

To a cooled solution of N,N′-carbonyldiimidazole (4.62 g, 28.5 mmol) in THF (30 mL) was added the (1S)-1-phenyl-1,2,3,4-tetrahydroisoquinoline (5.0 g, 23.9 mmol). The reaction mixture was stirred at room temperature for 2 hours.

Then, aluminium chloride (0.21 g, 1.6 mmol) was added and the mixture was stirred at room temperature for 10 minutes. A suspension of (3R)-3-quinuclidinol (compound V, 3.8 g, 29.9 mmol) in tetrahydrofuran (30 mL) and sodium hydride 60% (1.20 g, 30.0 mmol) was added in portions. The reaction mixture was refluxed for 3 h, and then was filtered and concentrated in vacuo. The obtained crude was suspended in water (100 mL) and aqueous solution of NaOH 10% was added until pH>10. The resulting aqueous phase was extracted with EtOAc (2×50 mL). The organic phase was then dried with sodium sulfate, filtered and evaporated to yield 6.92 g (mass corrected according to assay, 19.1 mmol, 80.0% yield, 98.22% HPLC purity) of solifenacin free base as an oil which was taken up in 43 mL of EtOAc. Separately, 2.20 g of succinic acid (18.62 mmol) and 45 mL of acetone were combined in a suitable reactor and heated to reflux until complete dissolution. The solution containing solifenacin base was then poured drop-wise to the heated succinic acid solution. The mixture was maintained at 55-60° C. for approximately 10-15 minutes with continuous stirring. The reactor was then cooled to room temperature and maintained at 20-25° C. for approximately 1 hour and then was cooled to 0-5° C. for 2 h.

Thereafter, the suspension was filtered, and the collected wet solid was dried under vacuum at 40° C. until constant weight to yield 7.91 g (16.46 mmol, 86.35%) of solifenacin succinate. Analysis by HPLC (area percentage): 99.45% of solifenacin, Titration: 100.07%.

### Example 3

### Preparation of (2-(1H-imidazole-2-ylcarbonyl)-(1S)-1-phenyl-1,2,3,4-tetrahydroisoquinoline

To a cooled suspension of N,N′-carbonyldiimidazole (6.8 g, 41.9 mmol) in dichloromethane (50 mL) (1S)-1-phenyl-1,2,3,4-tetrahydroisoquinoline (8.0 g, 38.2 mmol) was added. Once the solids dissolved, giving a slightly yellowish clear solution, the mixture was stirred at room temperature for 2 h. The reaction was quenched with water (50 ml), the organic layer was washed with water (2×25 ml), dried over Na₂SO₄, filtered and concentrated in vacuo to yield the carbamoylimidazole derivative of formula IV, named (2-(1H-imidazole-2-ylcarbonyl)-(1S)-1-phenyl-1,2,3,4-tetrahydroisoquinoline (12.3 g, quantitative yield, 99.3% HPLC) as an oil.

### Example 4

### Preparation of (2-(1H-imidazole-2-ylcarbonyl)-(1S)-1-phenyl-1,2,3,4-tetrahydroisoquinoline

To a cooled solution of N,N′-carbonyldiimidazole (6.8 g, 41.9 mmol) in THF (50 mL) (1S)-1-phenyl-1,2,3,4-tetrahydroisoquinoline (8.0 g, 38.2 mmol) was added. The reaction mixture was stirred at room temperature for 2 h. Removal of solvent under vacuum gave a viscous oil, which was dissolved in dichloromethane (50 mL) and washed with water (2×25 ml). The organic layer was dried over Na₂SO₄, filtered and concentrated in vacuo to yield the carbamoylimidazole derivative of formula IV, named (2-(1H-imidazole-2-ylcarbonyl)-(1S)-1-phenyl-1,2,3,4-tetrahydroisoquinoline (12.3 g, quantitative yield, 98.9% HPLC) as an oil.

### Example 5

### Preparation of (1S)-(3R)-1-azabicyclo[2.2.2]oct-3-yl-3,4-dihydro-1-phenyl-2(1H)-isoquinolinecarboxylate (solifenacin)

To the solution of (2-(1H-imidazole-2-ylcarbonyl)-(1S)-1-phenyl-1,2,3,4-tetrahydroisoquinoline (4 g, 13.2 mmol) in toluene (25 mL) titanium isopropoxide (0.5 mL, 1.8 mmol) was added. The mixture was stirred at room temperature for 30 minutes. Then, a suspension of (3R)-3-quinuclidinol (compound V, 2.13 g, 16.7 mmol) in toluene (25 mL) and sodium hydride 60% (0.67 g, 16.7 mmol) was added in portions. The reaction mixture was stirred at 35° C. for 6 h and refluxed for 4 additional hours. The reaction was quenched with water (25 ml), the obtained solid was filtered and decanted. The organic layer was dried over Na₂SO₄, filtered and concentrated in vacuo to yield a crude of solifenacin (analysis by HPLC (area percentage): 90.8% of solifenacin, 2.77% of (1S)-1-phenyl-1,2,3,4-tetrahydroisoquinoline).

### Example 6

### Preparation of (1S)-(3R)-1-azabicyclo[2.2.2]oct-3-yl-3,4-dihydro-1-phenyl-2(1H)-isoquinolinecarboxylate (solifenacin)

To the solution of (2-(1H-imidazole-2-ylcarbonyl)-(1S)-1-phenyl-1,2,3,4-tetrahydroisoquinoline (11.6 g, 38.2 mmol) in tetrahydrofuran (50 mL) titanium isopropoxide (1.45 mL, 4.9 mmol) was added. The mixture was stirred at room temperature for 30 minutes. Then, a suspension of (3R)-3-quinuclidinol (compound V, 6.0 g, 47.2 mmol) in tetrahydrofuran (50 mL) and sodium hydride 60% (1.85 g, 46.2 mmol) was added in portions. The reaction mixture was refluxed overnight. The reaction was quenched with brine (50 ml) and the obtained solid was filtered and decanted. The organic layer was dried over Na₂SO₄, filtered and concentrated in vacuo to yield a crude of solifenacin. Analysis by HPLC (area percentage): 90.7% of solifenacin, 1.14% of (1S)-1-phenyl-1,2,3,4-tetrahydroisoquinoline).

### Example 7

### Preparation of (1S)-(3R)-1-azabicyclo[2.2.2]oct-3-yl-3,4-dihydro-1-phenyl-2(1H)-isoquinolinecarboxylate butanedioate (1:1) (solifenacin succinate)

### (One Pot Procedure, Direct Addition)

To a cooled (0-5° C.) solution of 7.4 g (45.6 mmol) of N,N′-carbonyldiimidazole in THF (50 ml) (1S)-1-phenyl-1,2,3,4-tetrahydroisoquinoline (8.0 g, 3.82 mmol) was added. The mixture was left to reach room temperature and then stirred for 2 h to yield a clear solution. At this point, 1.45 ml of titanium isoproxyde (0.5 mmol) were added (Mixture A).

In parallel, a mixture consisting on 6 g (47.2 mmol) of (3R)-3-quinuclidinol (compound V), 50 ml of THF and 1.85 g (46 mmol) of NaH (60%) was prepared by stirring the mixture at room temperature for about 30 minutes (Mixture B).

Mixture B was added drop-wise over mixture A in about 15 minutes, then, the resulting mixture was refluxed for 10 hours, left to cool down, the inorganic salts filtered and the solvent evaporated. The resulting oil was dissolved in ethyl acetate and quenched with water. The organic phase was then extracted with diluted aqueous hydrochloric acid and rejected. The aqueous phase was then basified with potassium carbonate and extracted with ethyl acetate. The organic phase was then dried with sodium sulfate, filtered and evaporated to yield 9.68 g (26.7 mmol) of solifenacin free base as an oil which was taken up in 49.7 g (55 mL) of AcOEt and was heated to approximately 40-45° C. Separately, 3.15 g of succinic acid (26.70 mmol) and 35.5 g (45 mL) of acetone were combined in a suitable reactor and were heated to approximately 55-60° C. and maintained at this temperature with continuous stirring until complete dissolution. The solution containing solifenacin base was then poured into the heated succinic acid solution. The mixture was maintained at 55-60° C. for approximately 15-30 minutes with continuous stirring. The reactor was then cooled to room temperature and maintained at 20-25° C. for approximately 1 hour and then was cooled to 0-5° C. for 2 h.

Thereafter, the suspension was filtered, and the collected wet solid was dried under vacuum at 40° C. until constant weight to yield 10.73 g (22.33 mmol, 83.63%) of solifenacin succinate. Analysis by HPLC (area percentage): 97.76% of solifenacin.

### Example 8

### Preparation of (1S)-(3R)-1-azabicyclo[2.2.2]oct-3-yl-3,4-dihydro-1-phenyl-2(1H)-isoquinolinecarboxylate butanedioate (1:1) (solifenacin succinate)

### (One Pot Procedure, Inverted Addition)

To a cooled (0-5° C.) solution of 7.4 g (45.6 mmol) of N,N′-carbonyldiimidazole in THF (50 ml) (1S)-1-phenyl-1,2,3,4-tetrahydroisoquinoline (8.0 g, 3.82 mmol) was added. The mixture was left to reach room temperature and then stirred for 2 h to yield a clear solution. At this point, 1.45 ml of titanium isopropoxide (0.5 mmol) were added (Mixture A).

In parallel, a mixture consisting of 5.1 g (40.1 mmol) of (3R)-3-quinuclidinol ((compound V), 50 ml of THF and 1.61 g (40.3 mmol) of NaH (60%) was prepared by stirring the mixture at room temperature for about 30 minutes (Mixture B).

Mixture A was added drop-wise over mixture B in about 15 minutes, then, the resulting mixture was refluxed for 3 hours, left to cool down, the inorganic salts filtered and the solvent evaporated.

10.06 g (20.9 mmol) of solifenacin succinate were obtained by following the procedure described in example 5. Analysis by HPLC (area percentage): 98.86% of solifenacin. Potentiometric assay: 100.97%.

### Examples 9 to 12

### Effect of titanium isopropoxide

Following the procedure described in Example 8 for the preparation of the solifenacin free base, a set of experiments varying the amount of titanium isopropoxide was performed. The table below summarizes the results and shows the effect of the catalyst.

| | Amount of | |
|---|---|---|
| | titanium | % of compound II by |
| Example | isopropoxide⁽¹⁾ | HPLC⁽²⁾ |
| 9 | 0 | 33.23 |
| 10 | 0.36 | 8.53 |
| 11 | 0.73 | 1.61 |
| 12 | 1.45 | 1.39 |

⁽¹⁾as mol % of titanium isopropoxide referred to the molar amount of (1S)-1-phenyl-1,2,3,4-tetrahydroisoquinoline.
⁽²⁾area % by HPLC of compound II divided by the sum of the area % of compound II and solifenacin.

### Example 13

### Preparation of solifenacin succinate from solifenacin

Into a 250 ml three necked, rounded reaction vessel, equipped with a thermometer, addition funnel and distillation device, 4.29 g (1.3 equivalents) of succinic acid and 80.9 ml of acetone are charged. The mixture is refluxed to reach complete dissolution and 62.5 ml of isopropyl acetate solution of solifenacin base (1 equivalent) is added drop-wise while heating. 72 ml of solvent is distilled off, and the mixture is left to reach room temperature and further cooled in a water/ice slush for 2 h and filtered to obtain 12.23 g of solifenacin succinate. Yield: 87.91%, Assay: 99.78%.

It will be apparent to those skilled in the art that various modifications and variations can be made in the present invention and specific examples provided herein without departing from the spirit or scope of the invention. Thus, it is intended that the present invention covers the modifications and variations of this invention that come within the scope of any claims and their equivalents.

What is claimed is:

1. A process for making solifenacin (Ia), 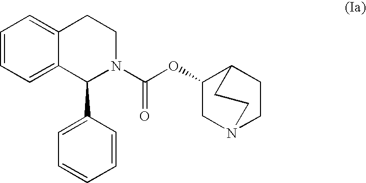
which process comprises reacting a compound of formula (IV) 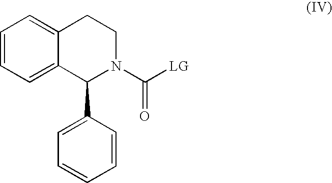
with a compound of formula V 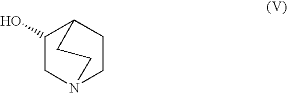
in the presence of at least one base, at least one Lewis acid compound and at least one organic solvent, wherein LG is a leaving group.

2. The process of claim 1, further comprising the step of converting the resulting solifenacin into one of its pharmaceutically acceptable salts.

3. The process of claim 2, wherein the pharmaceutically acceptable salt is a succinate salt.

4. The process of claim 1, wherein LG is 1H-imidazol-1-yl, 4-methyl-[1,2,4]oxadiazolidine-3,5-dione-2-yl, 1H-1,2,4-triazol-1-yl or trichloromethoxyl (OCCl₃).

5. The process of claim 4, wherein LG is 1H-imidazol-1-yl.

6. The process of claim 1, wherein the base is at least one of a hydride base, a C₁-C₁₀ alkoxide base, and a mixture thereof.

7. The process of claim 1, wherein the base is sodium hydride, sodium tert-amyloxide, or a mixture thereof.

8. The process of claim 7, wherein the base is sodium tert-amyloxide.

9. The process of claim 1, wherein the Lewis acid compound is at least one of a titanium based Lewis acid compound, an aluminium based Lewis acid compound, or a mixture thereof.

10. The process of claim 9, wherein the Lewis acid compound is titanium isopropoxide, aluminium trichloride, or a mixture thereof.

11. The process of claim 10, wherein the Lewis acid compound is aluminium trichloride.

12. The process of claim 1, wherein the organic solvent is an ether solvent, an aromatic hydrocarbon solvent, an aliphatic hydrocarbon solvent, or a mixture thereof.

13. The process of claim 12, wherein the organic solvent is tetrahydrofuran, 2-methyltetrahydrofuran, toluene, xylene, heptane, cyclohexane, or a mixture thereof.

14. The process of claim 13, wherein the organic solvent is tetrahydrofuran.

15. The process of claim 1, wherein the compound of formula (IV) is obtained by a process comprising reacting a compound of formula II 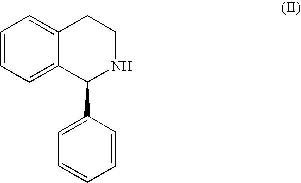
with a compound of formula III 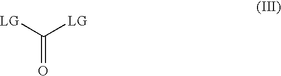
in the presence of at least one organic solvent,
wherein LG is a leaving group.

16. The process of claim 15, wherein the LG moieties of the compound of formula III are the same or different and are 1H-imidazol-1-yl, 4-methyl-[1,2,4]oxadiazolidine-3,5-dione-2-yl, 1H-1,2,4-triazol-1-yl or trichloromethoxyl (OCCl₃).

17. The process of claim 16, wherein each LG moiety of the compound of formula III is 1H-imidazol-1-yl.

18. The process of claim 15, wherein the organic solvent is an ether solvent, an aromatic hydrocarbon solvent, an aliphatic hydrocarbon solvent, or a mixture thereof.

19. The process of claim 18, wherein the organic solvent is tetrahydrofuran, 2-methyltetrahydrofuran, toluene, xylene, heptane, cyclohexane, or a mixture thereof.

20. The process of claim 19, wherein the organic solvent is tetrahydrofuran.

21. Solifenacin made according to the process of claim 1 or a pharmaceutically acceptable salt thereof.

22. The solifenacin or pharmaceutically acceptable salt of claim 21, having less than 5% of (S)-1-phenyl-1,2,3,4-tetrahydroisoquinoline (compound of formula II) or of a salt thereof.

23. The solifenacin or pharmaceutically acceptable salt of claim 21, having less than 2% of (S)-1-phenyl-1,2,3,4-tetrahydroisoquinoline (compound of formula II) or of a salt thereof.

24. The solifenacin or pharmaceutically acceptable salt of claim 21, having less than 1% of (S)-1-phenyl-1,2,3,4-tetrahydroisoquinoline (compound of formula II) or of a salt thereof.

25. The solifenacin or pharmaceutically acceptable salt of claim 21, having less than 0.5% of (S)-1-phenyl-1,2,3,4-tetrahydroisoquinoline (compound of formula II) or of a salt thereof.

26. The solifenacin or pharmaceutically acceptable salt of claim 21, having less than 0.2% of (S)-1-phenyl-1,2,3,4-tetrahydroisoquinoline (compound of formula II) or of a salt thereof.

27. A process for preparing solifenacin succinate salt, which process comprises:
(a) combining (i) a solution of solifenacin base in an ester solvent and (ii) a solution of succinic acid in a ketone solvent, to obtain a mixture comprising solifenacin succinate;
(b) allowing solifenacin succinate present in the resulting mixture to precipitate; and
(c) isolating precipitated solifenacin succinate from the mixture.

28. The process of claim 27, further comprising the step of partially distilling off the solvents from the mixture of step (a) to further concentrate the mixture.

29. The process of claim 27, wherein the ester solvent is ethyl acetate, isopropyl acetate, or a mixture thereof.

30. The process of claim 29, wherein the ester solvent is ethyl acetate.

31. The process of claim 27, wherein the ketone solvent is acetone, methyl ethyl ketone, or a mixture thereof.

32. The process of claim 31, wherein the ketone solvent is acetone.

33. The process of claim 3, wherein the solifenacin succinate salt is obtained according to the process of claim 27.

34. Solifenacin succinate salt made according to the process of claim 27.

35. A formulation comprising the solifenacin succinate salt of claim 34 and a pharmaceutically acceptable carrier.
